(19) United States
(12) Patent Application Publication
(10) Pub. No.: US 2019/0381708 A1
(43) Pub. Date: Dec. 19, 2019

(54) USING FILM TO PROVIDE A PREFORM FOR MICRO INJECTION MOLDING PROCESS

(71) Applicant: SABIC GLOBAL TECHNOLOGIES B.V., Bergen op Zoom (NL)

(72) Inventors: Johannes DE BROUWER, Oisterwijk (NL); Herwig JUSTER, Bergen Op Zoom (NL); Kenneth Francis LAWSON, Peterlee (GB)

(21) Appl. No.: 16/481,534

(22) PCT Filed: Jan. 31, 2018
(86) PCT No.: PCT/IB2018/050606

§ 371 Date: Jul. 29, 2019

Related U.S. Application Data
(60) Provisional application No. 62/452,625, filed on Jan. 31, 2017.

(51) Int. Cl. B29C 45/00; A61M 37/00; B29C 45/26

(52) U.S. Cl. CPC B29C 45/0046; A61M 37/0015; B29L 2031/7544; A61M 2207/10; A61M 2037/0053; B29C 45/263

(57) ABSTRACT

A mold assembly 100 for forming a microneedle array, wherein the mold assembly facilitates expulsion of trapped gasses from the molding system. The mold assembly may comprise a first mold portion 102 comprising a plurality of recesses 104 formed therein, a second mold portion 106 disposed adjacent the first mold portion, wherein a surface of the second mold portion and the plurality of recesses of the first mold portion define a mold cavity, a mold film insert 112 disposed within the mold cavity between the first mold portion and the second mold portion of the mold assembly, the mold film insert comprising a plurality of perforated layers 114, each of the perforated layers comprising a plurality of perforations.

### TECHNICAL FIELD

The application concerns forming microneedle arrays using a mold assembly for precise replication.

### BACKGROUND

Microneedles are attractive for delivery of certain therapeutics. These needles may be particularly desirable as a mode of therapeutic delivery because of the potential to replace syringe-with-needle type of injections with a pain free alternative. Microneedles can be virtually painless because they do not penetrate deep enough to contact nerves and only penetrate the outermost layer of the skin, unlike traditional syringes and hypodermic needles. Additionally, shallower penetration can also reduce the chance of infection or injury. Microneedles may also facilitate delivery of a more precise dosage of a therapeutic which enables the use of lower doses in treatments. Other advantages of microneedles for drug delivery include the simplified logistics (absence of required cold chain), ability for patient self-administration (no need for doctor, nurse, reduction of people transport). Beyond therapeutic delivery, drug delivery, microneedles have also been investigated for diagnostic applications. Bodily fluids coming out through the punctured skin can be analyzed for e.g. glucose or insulin.

Microneedles often require a manufacturing process that allows mass production at lowest cost, and as a consequence, shortest possible cycle time. In order to have proper transcription of mold texture and shape to the molded part, high flow may be necessary, especially having low viscosity at extremely high shear rates. Furthermore, good release from the production mold is important to reduce cycle time to improve the cost efficiency. These needles should have good strength to prevent breaking of the microneedle during usage. While there are a number of benefits to the use of microneedles and considerations with respect to forming them, certain challenges remain in microneedle production. It would be beneficial to prepare a process or system of replicating microneedles that exhibit an appropriate geometry for puncturing the skin.

### SUMMARY

Aspects of the present disclosure concern a mold assembly for forming a microneedle array, the mold assembly comprising: a first mold portion comprising a plurality of recesses formed therein; a second mold portion disposed adjacent the first mold portion, wherein a surface of the second mold portion and the plurality of recesses of the first mold portion define a mold cavity; and a mold film insert disposed within the mold cavity between the first mold portion and the second mold portion of the mold assembly, the mold film insert comprising a plurality of perforated layers, each of the perforated layers comprising a plurality of perforations, wherein a size of at least one of the plurality of perforations in at least two adjacent perforated layers varies between the adjacent perforated layers, and wherein at least a portion of the plurality of perforations of each of the adjacent perforated layers are configured to facilitate a flow of material through the mold film insert and into the mold cavity.

Other aspects concern methods of forming a microneedle array by heating a polymer to provide a molten polymer; and causing the molten polymer to move into a mold assembly and through a plurality of perforated layers disposed therein and into a plurality of recesses, wherein the perforated layers restrict flow of the molten polymer into at least the plurality of recesses and facilitates expulsion of trapped gases, wherein the plurality of perforated layers restrict flow of the molten polymer by filling a volume within the mold assembly except for space corresponding to the plurality of recesses in the mold assembly.

Further aspects relate to a microneedle array formed by a method comprising heating a polymer to provide a molten polymer; and causing the molten polymer to move through a plurality of perforated layers into a plurality of recesses, wherein the perforated layers restrict flow of the molten polymer gas and facilitate release of trapped gases from the plurality of recesses thereby allowing the molten polymer to flow therein.

### BRIEF DESCRIPTION OF DRAWINGS

The above-mentioned and other features and advantages of this disclosure, and the manner of attaining them, will become apparent and be better understood by reference to the following description of one aspect of the disclosure in conjunction with the accompanying drawings, wherein:

FIG. 1 depicts an exploded view of a mold assembly according to the present disclosure.

FIG. 2 depicts a schematic diagram of configurations for a plurality of perforations for a plurality perforated layers of the mold film insert.

FIG. 3 depicts a cross-sectional diagram of disclosure mold film insert of the present disclosure disposed within an injection molding apparatus.

### DETAILED DESCRIPTION OF ILLUSTRATIVE EMBODIMENTS

The present disclosure can be understood more readily by reference to the following detailed description of the disclosure. Microneedles can be used to deliver a therapeutic or to draw interstitial fluids or blood without penetrating tissue as deep as traditional needles. Such microneedles can be used individually or as an array of needles. The needles are typically produced via mass production at a low cost. To efficiently function as a therapeutic delivery mechanism or as a diagnostic tool, microneedles must be sufficiently sharp to penetrate dermal surfaces while still maintaining the benefit of being relatively pain free. Injection molding has been a means of mass production of microneedle arrays at low costs and high precision with respect to needle shape. Still, while injection molding production has its advantages, replication of the microneedle arrays can be disrupted because of variations in filling in the microneedle molds or microstructures. Injection molding small scale, specifically microscale parts as with microneedles, may be challenging because of the relatively large conduits throughout the mold. The relatively large conduits, compared to the microstructures of the mold, allow a substrate to flow much more freely than do the microstructure areas for shaping the microneedle array. The microstructure areas may restrict flow channels because of the significant surface to volume ratio in the microneedle structures. Gas may be trapped within mold cavities of the microstructure, thereby preventing filling. The trapped gasses may cause uneven filling of the microstructure which can result in variable length and inconsistent tips among the microneedles of the formed microneedle array. The mold assembly and methods of forming a microneedle array as described herein may provide a microneedle array having the desired geometry to provide a sharp tip among the microneedles and/or a sharp blade to properly penetrate or cut the skin. The mold assembly for forming a microneedle array may comprise a first mold portion, a second mold portion, and a mold film insert.

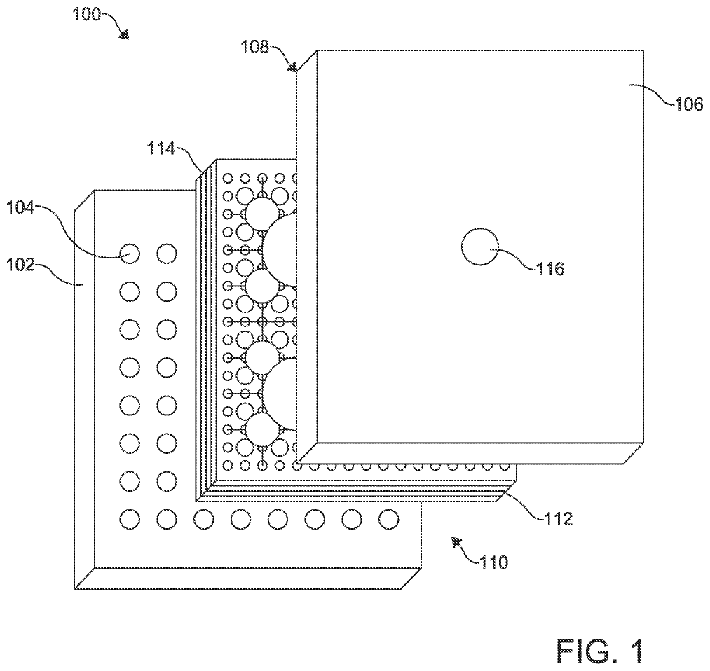

According to aspects of the present disclosure, the mold assembly may comprise a first mold portion comprising a plurality of recesses formed therein, a second mold portion, The first mold portion and the second mold portion may cooperate so that a surface of the second mold portion and the plurality of recesses of the first mold portion define a mold cavity at which the mold film insert is disposed. Referring to FIG. 1 showing an exploded view of a mold assembly 100, the first mold portion 102 may comprise a plurality of recesses 104 formed therein and corresponding to a configuration for a microneedle array. Within the mold assembly 100, a second mold portion 106 may be disposed adjacent the first mold portion 102. The second mold portion 106 may be disposed so that a surface 108 of the second mold portion 106 and the plurality of recesses 104 of the first mold portion 102 may cooperate to define a mold cavity 110. A mold film insert (also described as a mold insert) 112 comprising a plurality of perforated layers 114 may be disposed within the mold cavity 110. To allow a desired substrate into the mold cavity 110, the second mold portion may include a sprue, or channel 116.

A recess of the plurality of recesses of the first mold portion may have a particular geometry which may correspond to the shape of a microneedle in a microneedle array. At least a portion of the plurality of recesses may exhibit a half-pyramid geometry where two side lengths of the half-pyramid form an apex. The apex may correspond to a penetrative point, or tip, of a microneedle formed in the mold. Each recess may thus have a certain base size and apex, as well as an accompanying apex angle. In one example, the plurality of recesses may have a half pyramidal geometry with a base of 100 micrometer (μm) and a side length of 250 μm. In further examples, at least a portion of the plurality of recesses may vary in size relative to each other. This variation in size creates a varying aspect ratio in the microneedle array. For example, side lengths of the half-pyramid geometry of each recess may vary.

According to aspects of the present disclosure, a mold film insert may be disposed within the mold cavity. The mold film insert may be disposed between the first mold portion and the second mold portion of the mold assembly. The mold film insert may comprise a plurality of perforated layers. Each layer of the plurality of perforated layers may comprise a plurality of perforations. The plurality of perforated layers of the mold film insert is configured to affect a melt flowpath of a substrate, generally a molten polymer, into the mold cavity formed by the first mold portion and the second mold portion. A size of at least one of the plurality of perforations in at least two adjacent perforated layers may vary between the adjacent perforated layers.

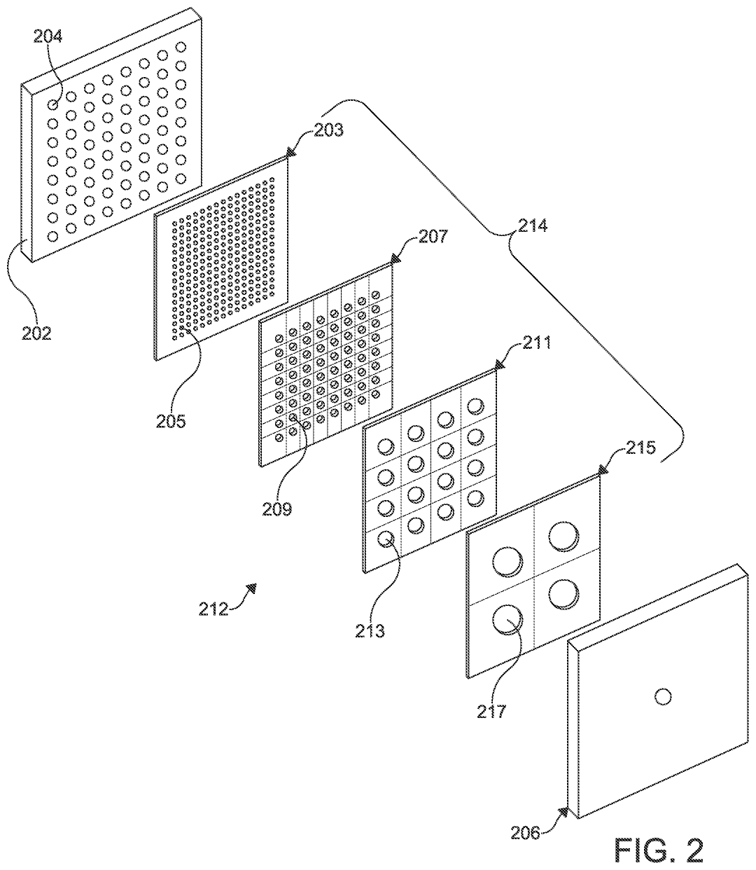

FIG. 2 describes a configuration for the plurality of perforated layers of the mold film insert presented as exemplary individual layers with the first portion of the mold assembly. A first perforated layer 203 of a mold film insert 212 may comprise a first plurality of perforations 205 of a similar size and similar population to a plurality of recesses 204 of a first mold portion 202. Thus, the first perforated layer 203 of the plurality of perforated layers 214 is situated closest to the first mold portion 202 of the mold assembly 200 relative to remaining perforated layers of the plurality of perforated layers 214 comprising the mold film insert 212. More specifically, the mold film insert 212 may be oriented so that the first perforated layer 203 of the perforated layers 214 is adjacent a plurality of recesses 204 of the first mold portion 202. A second mold portion 206 is adjacent the first mold portion 202 with the plurality of perforated layers 214 there between.

As described, the first perforated layer 203 of the mold film insert 212 may comprise perforations of a similar size and similar population to the plurality of recesses 204 of the first mold portion 202. Specifically, the first perforated layer 203 of the mold film insert may comprise perforations of a similar size to that of an apex of a recess of the plurality of recesses 204 of the first mold portion 202. In one example, the first perforated layer 203 may comprise a mesh or woven material having perforations or apertures throughout the layer. The perforations or apertures throughout the mesh or woven material may be of a similar size to that of the apex of a recess of the plurality of recesses 204 of the first mold portion 202, where the apex may correspond to the tip of a microneedle.

A second perforated layer 207 may be disposed adjacent the first perforated layer 203 forming the plurality of perforated layers 214 of the mold film insert 212. In the mold assembly 200, the second perforated layer 207 may be disposed adjacent the first perforated layer 203 towards a second mold portion of the mold assembly 206. The second perforated layer 207 may comprise a second plurality of perforations 209. At least a portion of the perforations of the second plurality of perforations 209 may have a size similar to that of a diameter of a recess of the plurality of recesses 204 of the first mold portion 202. In an example, perforations of the second perforated layer 207 may have a grid-like configuration.

A third perforated layer 211 may be disposed adjacent the second perforated layer 207. In the mold assembly 212, the third perforated layer 211 may be disposed adjacent the second perforated layer 207 and oriented towards the second mold portion 206. The third perforated layer 211 may comprise a third plurality of perforations 213. At least a portion of the perforations of the third plurality of perforations 213 may be sized so as to be about twice the size of at least a portion of the second plurality of perforations 209 of the second perforated layer 207. Thus, the third perforated layer 211 may have fewer perforations than the second perforated layer 207. Perforations of the third perforated layer 211 may be spaced at a distance about two times a distance of that between at least a portion of the second plurality of perforations 209 of the second perforated layer 207. As an example, perforations of the third perforated layer 211 may have a grid-like configuration so that perforations of the third perforated layer 211 may be spaced at a distance about two times a distance of that between at least a portion of the second plurality of perforations 211 of the second perforated layer 207.

A fourth perforated layer 215 may be disposed adjacent the third perforated layer 211. In the mold assembly 212, the fourth perforated layer 215 may be disposed adjacent the third perforated layer 211 towards the second mold portion 206. The fourth perforated layer 215 may comprise a fourth plurality of perforations 217. At least a portion of the fourth plurality perforations 217 of the fourth perforated layer 215 may be sized so as to be about twice the size of at least a portion of the third plurality of perforations 213 of the third perforated layer 211. Thus, the fourth perforated layer 215 may have fewer perforations than the third perforated layer 211. Perforations of the fourth perforated layer 215 may be spaced at a distance about two times a distance of that between at least a portion of the third plurality perforations 213 of the third perforated layer 211. For example, perforations of the fourth perforated layer 215 may have a grid-like configuration so that perforations of the fourth perforated layer 215 may be spaced at a distance about two times a distance of that between at least a portion of the third plurality of perforations 213 of the third perforated layer 211.

Each perforated layer of the plurality of perforated layers may comprise a plurality of perforations. These perforations may be formed in the surface of a given layer. All of the perforations may be formed at the same surface of a layer. The layer may comprise a film. The film may be readily perforated.

Further perforated layers may be useful to accommodate the size of a microneedle array. Generally, as the number position of perforated layers increases, so does the size of the perforations. The number of perforations may decrease. In various aspects, the described geometry of the mold film insert comprising the disclosed plurality of perforated layers may alter the flow path of a substrate (i.e., a polymer) that has been introduced to the mold assembly. The plurality of perforated layers may restrict a distributed flow path to the plurality of recesses of the first mold portion when the mold assembly is assembled having the mold film insert disposed between the first and second mold portions.

Flowpath of a substrate, such as a molten polymer, may be restricted in the mold cavity by the plurality of perforated layers of the mold film insert. A substrate, such as a polymer, may be heated to provide a molten polymer. The molten polymer may be caused to flow a mold assembly as described herein. Within the mold assembly, the molten polymer may proceed through a plurality of perforated layers disposed therein and into a plurality of recesses, wherein the perforated layers restrict the flowpath (flow) of the molten polymer into at least the plurality of recesses and facilitates expulsion of trapped gases. The plurality of perforated layers may restrict the flowpath of the molten polymer by filling a volume within the mold assembly except for space corresponding to the plurality of recesses in the mold assembly. Passage of the substrate through the plurality of perforated layers may form a base for the formed microneedle array. According to methods of the present disclosure, the mold assembly may be disposed within an injection molding apparatus. Thus, flowpath of a substrate may be restricted in the disclosed mold assembly where the mold assembly is disposed within an injection molding apparatus.

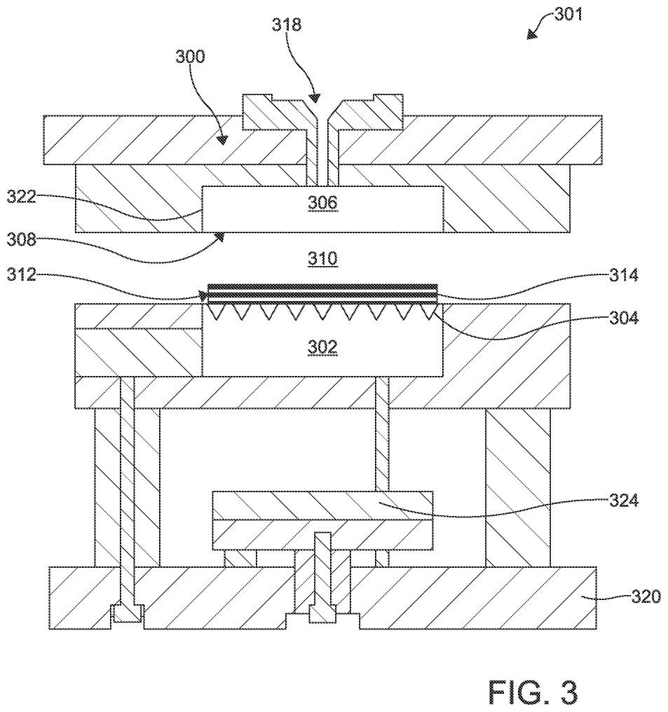

A basic injection molding apparatus may comprise, for example, an ejector system, to facilitate demolding of a molded part (here, a microneedle) from the mold assembly; a stationary side, to hold portions of the mold assembly, a moving side to bring portions of the mold assembly into contact; and a sprue, to allow passage of a substrate into the mold assembly. As shown in FIG. 3, a mold assembly 300 of the present disclosure may be disposed within an injection molding apparatus 301. Within the mold assembly 300, a second mold portion 306 may be disposed adjacent a first mold portion 302 comprising a plurality of recesses 304. The second mold portion 306 may be disposed so that a surface 308 of the second mold portion 306 and the plurality of recesses 304 of the first mold portion 302 may cooperate to define a mold cavity 310. A mold film insert 312 comprising a plurality of perforated layers 314 may be disposed within the mold cavity 310.

In the injection molding apparatus 301, a substrate may be contacted with the mold cavity 310 of the mold assembly 300. During operation, the first and second mold portions 302, 306 may be engaged so that the first and second mold portions are contacted. Contacting of the first and second mold portions 302, 306 encloses the mold cavity 310 and the mold film insert 312 disposed therein. Engagement of the first and second mold portions 302, 306 may be performed by a moving side 320 of the injection molding apparatus 301 operating to meet a stationary side 322 of the injection molding apparatus 301. In some aspects, the first mold portion 302 of the mold assembly 300 may be disposed within the moving side 320 of the injection molding apparatus 301 and the second mold portion 306 may be disposed within the stationary side 322 of the injection molding apparatus 301.

A substrate, such as a molten polymer, may enter the engaged mold assembly 300 via a sprue 318, or channel As the substrate enters the mold assembly 300, the substrate contacts the mold cavity 310 and the mold film insert 312 disposed therein. By force of the substrate entering the mold cavity 310 via the sprue 318, the substrate may be displaced into at least a portion of the perforations of the plurality of perforated layers 314 of the mold film insert 312. Specifically, a substrate as a molten polymer may be caused to move through the plurality of perforated layers 314 of the mold film insert 312 and then flow into the plurality of recesses 304 of the first mold portion 302. The plurality of perforated layers 314 may restrict flow of the molten polymer gas and facilitate release of trapped gases from the plurality of recesses 302 thereby allowing the molten polymer to flow therein forming a molded part. An ejector system 324 may be engaged to demold the molded part from the mold assembly. As such, the disclosed geometries of the perforated layers described herein facilitate replication of a mold part, specifically, a microneedle array having microneedles corresponding to the plurality of recesses of the first mold portion. Because the disclosed geometries of the perforated layers facilitates an expulsion of trapped gasses within the mold assembly in the injection molding apparatus, the mold assembly of the present disclosure may provide a more precise replication generally required for the formation of microstructures of the microneedle array.

Individual perforated layers of the mold film insert of the present disclosure may be formed from a film. The film may comprise an extruded film for example. To form the desired perforations, the film may be bored by a machining process. Where a film is bored, these perforations may be formed in the surface of a given layer. All of the perforations may be formed at the same surface of a layer. In further aspects, the plurality of perforated layer of the mold film insert may be manufactured by an appropriately precise process to facilitate perforations. The plurality of perforated layers may be manufactured precisely according to a low voltage electrical discharge machining (EDM) process.

The perforated layers of the mold film insert may comprise a polymeric material. In some examples, the perforated layers may comprise the same material as the substrate for forming the microneedle array. In further examples, the perforated layers may comprise a material that is similar to the substrate for forming the microneedle array with respect to properties such as flow, viscosity, melting temperature, or glass transition temperature, for example. As provided herein, the substrate enters the mold assembly and fills the perforations of the perforated layers through which the flowpath of the substrate is limited. The substrate fills the perforations thereby forming a base for the microneedle array within the mold assembly. As an example, heat within the molding apparatus forms the perforated layers and substrate together to form the base.

In certain aspects, at least a portion of the plurality of recesses of the first mold portion may vary in size relative to each other. This variation in size creates a varying aspect ratio in the microneedle array. For example, side lengths of the half-pyramid geometry of each laminate cavity may vary. At least a first portion of the plurality of recesses may have a side length of up to about 0.8 millimeters (mm) while at least a second portion of the plurality of recesses may have side length of up to about 1.0 mm. The varying side lengths of the plurality of recesses may ensure that the base size of the plurality of recesses also varies. Over the variation of the bases, different aspect ratios from about 1:2 to about 1:4, or from 1:2 to 1:4, may be apparent within the microneedle array. The varying aspect ratios may allow for different cutting or penetrative profiles of microneedles formed using the mold assembly described herein.

As provided, microneedles of the microneedle array formed using the mold assembly of the present disclosure may be used to deliver a therapeutic or to draw interstitial fluids or blood without penetrating tissue as deep a traditional needles. The microneedles may be used individually or as an array of needles. The size of such needles typically is measured in microns. Some microneedles are between 100 μm and 1 mm in length, preferably between 10 μm and 500 μm, more preferably between 30 μm and 200 μm and more preferably between 100 μm and 150 μm. The needles are typically produced via mass production at a low cost. To function efficiently as a therapeutic delivery mechanism or as a diagnostic tool, microneedles must be sufficiently sharp to penetrate dermal surfaces while still maintaining the benefit of being relatively pain free. Thus, a given microneedle production array is desired to exhibit a certain aspect ratio among the formed microneedles while the formed needles still maintain their structural integrity and strength.

The mold assembly and methods of forming thereof may provide a microneedle array having the desired geometry sufficient to provide a sharp tip among the microneedles and a sharp blade to properly first mold portion, a second mold portion, and a mold film insert. The first mold portion may comprise a plurality of recesses, each of the recesses having a half pyramid geometry. The plurality of recesses may cooperate with a surface of the second mold portion to define a mold cavity in which the mold film insert is disposed. The mold film insert may comprise a plurality of perforated layers that may alter the flowpath of a substrate introduced to the mold assembly.

A microneedle array as formed in the present disclosure may comprise solid microneedles. In an aspect, for therapeutic delivery via a solid microneedle array, the therapeutic may be coated onto the microneedles and dissolves or diffuses. That is, active components of the therapeutic may dissolve or diffuse when the microneedles penetrate skin, allowing interstitial fluid to contact the drug formulation. In this way, the therapeutic may be released just below the skin. Microneedles formed herein should have sufficient mechanical strength to remain intact (i) while being inserted into the biological barrier, (ii) while remaining in place for up to a number of days, and (iii) while being removed.

Furthermore, chemical resistance of the microneedle array may fulfill regulatory critical to quality (CTQ) requirements. There should be minimal or no chemical reaction among the active ingredient of the therapeutic, the carrier/coating, and the material forming the microneedle array during production, sterilization, storage, and/or during the use of the microneedle array. Such interactions may destroy or alter the active ingredient, affect needle properties, or both. In various aspects, the microneedle array formed according to the methods described herein exhibit both the strength and ductility that may be lacking in conventional microneedle arrays.

Microneedles may be manufactured via commercial molding technology. A microneedle array may be formed using the mold assembly of the present disclosure. In one aspect, the mold assembly may be inserted in a conventional injection molding apparatus for forming a microneedle array. As an example, the mold assembly may be inserted into an injection molding apparatus. In a single injection molding cycle, a microneedle array may be formed. The plurality of perforated layers of the mold film insert may allow a user to improve replication of needles by improving how a substrate fills the recesses of the mold assembly that serve as the microstructure for the microneedle array mold.

In various aspects, the substrate may comprise a polymer material. The substrate for forming a microneedle array using the disclosed mold assembly may comprise a polymer or a mixture of polymers. Generally, the polymer mixture may be supplied in a liquid or flowable state, via for example, an extrusion die apparatus, to the mold assembly. A solid product comprising the microneedle array may then be obtained from the mold assembly after cooling. Exemplary polymer materials may comprise engineering thermoplastics such as polycarbonates, polyetherimides, polyphenylene ether, liquid crystalline polymers and polybutylene terephthalate, as well as blends of polycarbonate with acrylic (or acrylonitrile) butadiene styrene plastics.

The substrate may comprise a polycarbonate. The terms “polycarbonate” or “polycarbonates” as used herein includes copolycarbonates, homopolycarbonates and (co)polyester carbonates. The term polycarbonate can be further defined as compositions have repeating structural units of the formula (1):

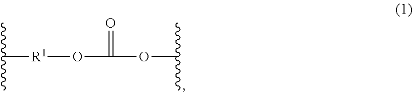

in which at least 60 percent of the total number of IV groups are aromatic organic radicals and the balance thereof are aliphatic, alicyclic, or aromatic radicals. In a further aspect, each IV is an aromatic organic radical and, more preferably, a radical of the formula (2):

-A¹-Y¹-A² (2),

wherein each of A¹ and A² is a monocyclic divalent aryl radical and Y¹ is a bridging radical having one or two atoms that separate A¹ from A². In various aspects, one atom separates A¹ from A². For example, radicals of this type include, but are not limited to, radicals such as —O—, —S—, —S(O)—, 1'S(O₂)—, —C(O)—, methylene, cyclohexyl-methylene, 2-[2.2.1]-bicycloheptylidene, ethylidene, isopropylidene, neopentylidene, cyclohexylidene, cyclopentadecylidene, cyclododecylidene, and adamantylidene. The bridging radical Y¹ is preferably a hydrocarbon group or a saturated hydrocarbon group such as methylene, cyclohexylidene, or isopropylidene. Polycarbonate materials include materials disclosed and described in U.S. Pat. No. 7,786,246, which is hereby incorporated by reference in its entirety for the specific purpose of disclosing various polycarbonate compositions and methods for manufacture of same. Polycarbonate polymers can be manufactured by means known to those skilled in the art.

An exemplary polymer of the present disclosure may include additives such as a mold release agent to facilitate ejection of a formed microneedle array from the mold assembly. Examples of mold release agents include both aliphatic and aromatic carboxylic acids and their alkyl esters, for example, stearic acid, behenic acid, pentaerythritol tetrastearate, glycerin tristearate, and ethylene glycol distearate. Polyolefins such as high-density polyethylene, linear low-density polyethylene, low-density polyethylene, and similar polyolefin homopolymers and copolymers can also be used a mold release agents. Some compositions use pentaerythritol tetrastearate, glycerol monostearate, a wax or a poly alpha olefin. Mold release agents are typically present in the composition at 0.05 to 10 wt %, based on total weight of the composition, specifically 0.1 to 5 wt %, 0.1 to 1 wt % or 0.1 to 0.5 wt %. Some preferred mold release agents will have high molecular weight, typically greater than 300, to prevent loss of the release agent from the molten polymer mixture during melt processing.

The polymer material for forming the microneedle array may further comprise one or more additives intended to impart certain characteristics to a microneedle array formed by the mold assembly described herein. The polymer material may include one or more of an impact modifier, flow modifier, antioxidant, heat stabilizer, light stabilizer, ultraviolet (UV) light stabilizer, UV absorbing additive, plasticizer, lubricant, antistatic agent, antimicrobial agent, colorant (e.g., a dye or pigment), surface effect additive, radiation stabilizer, or a combination comprising one or more of the foregoing. For example, a combination of a heat stabilizer, and ultraviolet light stabilizer can be used. In general, the additives are used in the amounts generally known to be effective. For example, the total amount of the additive composition can be 0.001 to 10.0 wt %, or 0.01 to 5 wt %, each based on the total weight of all ingredients in the composition.

The polymer material may include various additives ordinarily incorporated into polymer compositions, with the proviso that the additive(s) are selected so as to not significantly adversely affect the desired properties of the thermoplastic composition (good compatibility for example). Such additives can be mixed at a suitable time during the mixing of the components for forming the composition.

In addition, the polymer material may exhibit excellent release, as measured by ejection force (N) and coefficient of friction. The polymer material also preferably show (i) high flow at high shear conditions to allow good transcription of mold texture and excellent filling of the finest mold features, (ii) good strength and impact (as indicated by ductile Izod Notched Impact at room temperature and modulus), and (iii) high release to have efficient de-molding and reduced cooling and cycle time during molding. The microneedles formed herein may have sufficient mechanical strength to remain intact (i) while being inserted into the biological barrier, (ii) while remaining in place for up to a number of days, and (iii) while being removed.

Definitions

It is to be understood that the terminology used herein is for the purpose of describing particular aspects only and is not intended to be limiting. As used in the specification and in the claims, the term “comprising” can include the embodiments “consisting of” and “consisting essentially of” Unless defined otherwise, all technical and scientific terms used herein have the same meaning as commonly understood by one of ordinary skill in the art to which this disclosure belongs. In this specification and in the claims which follow, reference will be made to a number of terms which shall be defined herein.

As used in the specification and the appended claims, the singular forms “a,” “an” and “the” include plural equivalents unless the context clearly dictates otherwise. Thus, for example, reference to “a polycarbonate polymer” includes mixtures of two or more polycarbonate polymers.

Ranges can be expressed herein as from one value (first value) to another value (second value). When such a range is expressed, the range includes in some aspects one or both of the first value and the second value. Similarly, when values are expressed as approximations, by use of the antecedent ‘about,’ it will be understood that the particular value forms another aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the terms “about” and “at or about” mean that the amount or value in question can be the designated value, approximately the designated value, or about the same as the designated value. It is generally understood, as used herein, that it is the nominal value indicated ±5% variation unless otherwise indicated or inferred. The term is intended to convey that similar values promote equivalent results or effects recited in the claims. That is, it is understood that amounts, sizes, formulations, parameters, and other quantities and characteristics are not and need not be exact, but can be approximate and/or larger or smaller, as desired, reflecting tolerances, conversion factors, rounding off, measurement error and the like, and other factors known to those of skill in the art. In general, an amount, size, formulation, parameter or other quantity or characteristic is “about” or “approximate” whether or not expressly stated to be such. It is understood that where “about” is used before a quantitative value, the parameter also includes the specific quantitative value itself, unless specifically stated otherwise.

Disclosed are the components to be used to prepare the compositions of the disclosure as well as the compositions themselves to be used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the disclosure. Thus, if there are a variety of additional steps that can be performed it is understood that each of these additional steps can be performed with any specific aspect or combination of aspects of the methods of the disclosure.

References in the specification and concluding claims to parts by weight, of a particular element or component in a composition or article, denotes the weight relationship between the element or component and any other elements or components in the composition or article for which a part by weight is expressed. Thus, in a compound containing 2 parts by weight of component X and 5 parts by weight component Y, X and Y are present at a weight ratio of 2:5, and are present in such ratio regardless of whether additional components are contained in the compound.

As used herein the terms “weight percent,” “weight %,” and “wt. %” of a component, which can be used interchangeably, unless specifically stated to the contrary, are based on the total weight of the formulation or composition in which the component is included. For example if a particular element or component in a composition or article is said to have 8% by weight, it is understood that this percentage is relative to a total compositional percentage of 100% by weight.

As used herein, the terms “weight average molecular weight” or “Mw” can be used interchangeably, and are defined by the formula:

$${M}_{w} = \frac{\sum {N}_{i} {{M}_{i}}^{2}}{\sum {N}_{i} {M}_{i}} ,$$

where Mi is the molecular weight of a chain and Ni is the number of chains of that molecular weight. Mw can be determined for polymers, e.g. polycarbonate polymers, by methods well known to a person having ordinary skill in the art using molecular weight standards, e.g. polycarbonate standards or polystyrene standards, preferably certified or traceable molecular weight standards. Polystyrene basis refers to measurements using a polystyrene standard.

The term “siloxane” refers to a segment having a Si—O—Si linkage.

The term “flowable” means capable of flowing or being flowed. Typically a polymer is heated such that it is in a melted state to become flowable. ° C. is degrees Celsius. ˜m is micrometer.

Aspects

The present disclosure comprises at least the following aspects.

Aspect 1A. A mold assembly for forming a microneedle array, the mold assembly comprising: a first mold portion comprising a plurality of recesses formed therein; a second mold portion disposed adjacent the first mold portion, wherein a surface of the second mold portion and the plurality of recesses of the first mold portion define a mold cavity; and a mold film insert disposed within the mold cavity between the first mold portion and the second mold portion of the mold assembly, the mold film insert comprising a plurality of perforated layers, each of the perforated layers comprising a plurality of perforations, wherein a size of at least one of the plurality of perforations in at least two adjacent perforated layers varies between the adjacent perforated layers, and wherein at least a portion of the plurality of perforations of each of the adjacent perforated layers are configured to facilitate a flow of material through the mold film insert and into the mold cavity.

Aspect 1B. A mold assembly for forming a microneedle array, the mold assembly consisting essentially of: a first mold portion comprising a plurality of recesses formed therein; a second mold portion disposed adjacent the first mold portion, wherein a surface of the second mold portion and the plurality of recesses of the first mold portion define a mold cavity; and a mold film insert disposed within the mold cavity between the first mold portion and the second mold portion of the mold assembly, the mold film insert comprising a plurality of perforated layers, each of the perforated layers comprising a plurality of perforations, wherein a size of at least one of the plurality of perforations in at least two adjacent perforated layers varies between the adjacent perforated layers, and wherein at least a portion of the plurality of perforations of each of the adjacent perforated layers are configured to facilitate a flow of material through the mold film insert and into the mold cavity.

Aspect 1C. A mold assembly for forming a microneedle array, the mold assembly consisting of: a first mold portion comprising a plurality of recesses formed therein; a second mold portion disposed adjacent the first mold portion, wherein a surface of the second mold portion and the plurality of recesses of the first mold portion define a mold cavity; and a mold film insert disposed within the mold cavity between the first mold portion and the second mold portion of the mold assembly, the mold film insert comprising a plurality of perforated layers, each of the perforated layers comprising a plurality of perforations, wherein a size of at least one of the plurality of perforations in at least two adjacent perforated layers varies between the adjacent perforated layers, and wherein at least a portion of the plurality of perforations of each of the adjacent perforated layers are configured to facilitate a flow of material through the mold film insert and into the mold cavity.

Aspect 2. The mold assembly of aspects 1A-1C, wherein a first perforated layer of the plurality of perforated layers has perforations similar in size to that of at least a portion of a recess of the plurality of recesses.

Aspect 3. The mold assembly of aspect 2, wherein a second perforated layer is disposed at a surface of a first perforated layer and wherein the second perforated layer has perforations half the size of first layer perforations, wherein a third perforated layer is disposed at a surface of the second perforated layer and wherein the third perforated layer has perforations half the size of the second layer perforations, and wherein a fourth layer is disposed at a surface of the third perforated layer and wherein the fourth perforated layer has perforations half the size of the third layer perforations.

Aspect 4. The mold assembly of aspect 3, wherein the perforations of the second layer are spaced twice as far a part in the second perforated layer compared to the perforations of the first layer.

Aspect 5. The mold assembly of any one of aspects 3-4, wherein the perforations of the third perforated layer are spaced twice as far apart as the perforations of the second layer.

Aspect 6. The mold assembly of any one of aspects 3-5, wherein the perforations of the fourth layer are spaced twice as far the perforations of the third perforated layer.

Aspect 7. The mold assembly of any one of aspects 1A-6, wherein the plurality of perforated layers comprises polymer layers.

Aspect 8. The mold assembly of any one of aspects 1A-6, wherein the plurality of perforated layers comprises a second material that is the same as or similar to the material flowing through the mold insert.

Aspect 9. The mold assembly of any one of aspects 1A-8, wherein the plurality of perforated layers is formed from a multilayer sheet or a multilayer film.

Aspect 10. The mold assembly of any one of aspects 1A-9, wherein the mold assembly is part of injection molding system.

Aspect 11. The mold assembly of any one of aspects 1A-10, wherein at least a recess of the plurality of recesses comprises a half pyramidal geometry.

Aspect 12A. A method of forming a microneedle array, the method comprising: heating a polymer to provide a molten polymer; and causing the molten polymer to move into a mold assembly and through a plurality of perforated layers disposed therein and into a plurality of recesses, wherein the perforated layers restrict flow of the molten polymer into at least the plurality of recesses and facilitates expulsion of trapped gases, wherein the plurality of perforated layers restrict flow of the molten polymer by filling a volume within the mold assembly except for space corresponding to the plurality of recesses in the mold assembly.

Aspect 12B. A method of forming a microneedle array, the method consisting essentially of: heating a polymer to provide a molten polymer; and causing the molten polymer to move into a mold assembly and through a plurality of perforated layers disposed therein and into a plurality of recesses, wherein the perforated layers restrict flow of the molten polymer into at least the plurality of recesses and facilitates expulsion of trapped gases, wherein the plurality of perforated layers restrict flow of the molten polymer by filling a volume within the mold assembly except for space corresponding to the plurality of recesses in the mold assembly.

Aspect 12C. A method of forming a microneedle array, the method consisting of: heating a polymer to provide a molten polymer; and causing the molten polymer to move into a mold assembly and through a plurality of perforated layers disposed therein and into a plurality of recesses, wherein the perforated layers restrict flow of the molten polymer into at least the plurality of recesses and facilitates expulsion of trapped gases, wherein the plurality of perforated layers restrict flow of the molten polymer by filling a volume within the mold assembly except for space corresponding to the plurality of recesses in the mold assembly.

Aspect 13. The method of any of aspects 12A-12C, wherein the plurality of perforated layers forms a mold insert disposed within the mold assembly.

Aspect 14. The method of any one of aspects 12A-13, wherein the plurality of recesses is configured to form a microneedle array.

Aspect 15. The method of any one of aspects 12A-14, wherein the polymer comprises a polycarbonate.

Aspect 16. The method of any one of aspects 12A-14, wherein the plurality of perforated layers comprise a polycarbonate.

Aspect 17. The method of any one of aspects 12-16, wherein the plurality of perforated layers and the polymer comprise a same or similar polymeric material or combination of polymeric materials.

Aspect 18. The method of any one of aspects 12A-14, wherein the polymer and the plurality of perforated layers comprise a polycarbonate.

Aspect 19. The method of any one of aspects 12A-18, wherein the molten polymer fills at least a portion of the plurality of perforations of the plurality of perforated layers forming a base for the microneedle array.

Aspect 20. The method of aspect 19, wherein the base comprises the plurality of perforated layers and the polymer therein.

Aspect 21A. A microneedle array formed by a method comprising: heating a polymer to provide a molten polymer; and causing the molten polymer to move through a plurality of perforated layers into a plurality of recesses, wherein the perforated layers restrict flow of the molten polymer gas and facilitate release of trapped gases from the plurality of recesses thereby allowing the molten polymer to flow therein.

Aspect 21B. A microneedle array formed by a method consisting essentially of: heating a polymer to provide a molten polymer; and causing the molten polymer to move through a plurality of perforated layers into a plurality of recesses, wherein the perforated layers restrict flow of the molten polymer gas and facilitate release of trapped gases from the plurality of recesses thereby allowing the molten polymer to flow therein.

Aspect 21C. A microneedle array formed by a method consisting of: heating a polymer to provide a molten polymer; and causing the molten polymer to move through a plurality of perforated layers into a plurality of recesses, wherein the perforated layers restrict flow of the molten polymer gas and facilitate release of trapped gases from the plurality of recesses thereby allowing the molten polymer to flow therein.

Aspect 22. The microneedle array of aspect 21A, wherein the plurality of recesses correspond to the configuration of a microneedle array.

Aspect 23. The microneedle array of any one of aspects 21A-22, wherein the plurality of perforated layers provides a preformed base for the microneedle array as the molten polymer is caused to move through the plurality of perforated layers.

Aspect 24. The microneedle array of any one of aspects 21A-22, wherein the plurality of perforated layers forms a base for the microneedle array as the molten polymer is caused to move through the plurality of perforated layers.

It will be apparent to those skilled in the art that various modifications and variations can be made in the present disclosure without departing from the scope or spirit of the disclosure. Other aspects of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

The patentable scope of the disclosure is defined by the claims, and can include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A mold assembly for forming a microneedle array, the mold assembly comprising:
a first mold portion comprising a plurality of recesses formed therein;
a second mold portion disposed adjacent the first mold portion, wherein a surface of the second mold portion and the plurality of recesses of the first mold portion define a mold cavity; and
a mold film insert disposed within the mold cavity between the first mold portion and the second mold portion of the mold assembly, the mold film insert comprising a plurality of perforated layers, each of the perforated layers comprising a plurality of perforations,
wherein a size of at least one of the plurality of perforations in at least two adjacent perforated layers varies between the adjacent perforated layers, and
wherein at least a portion of the plurality of perforations of each of the adjacent perforated layers are configured to facilitate a flow of material through the mold film insert and into the mold cavity.

2. The mold assembly of claim 1, wherein a first perforated layer of the plurality of perforated layers has perforations similar in size to that of at least a portion of a recess of the plurality of recesses.

3. The mold assembly of claim 2, wherein a second perforated layer is disposed at a surface of a first perforated layer and wherein the second perforated layer has second layer perforations that are half the size of first layer perforations, wherein a third perforated layer is disposed at a surface of the second perforated layer and wherein the third perforated layer has perforations half the size of second layer perforations, and wherein a fourth perforated layer is disposed at a surface of the third perforated layer and wherein the fourth perforated layer has perforations half the size of the third layer perforations.

4. The mold assembly of claim 3, wherein the perforations of the second perforated layer are spaced twice as far a part in the second perforated layer compared to the perforations of the first layer.

5. The mold assembly of claim 3, wherein the perforations of the third perforated layer are spaced twice as far apart as the perforations of the second perforated layer.

6. The mold assembly of claim 3, wherein the perforations of the fourth perforated layer are spaced twice as far perforations of the third layer.

7. The mold assembly of claim 1, wherein the plurality of perforated layers comprises polymer layers.

8. The mold assembly of claim 1, wherein the plurality of perforated layers comprises a second material that is the same as or similar to the material flowing through the mold insert.

9. The mold assembly of claim 1, wherein the plurality of perforated layers is formed from a multilayer sheet or a multilayer film.

10. The mold assembly of claim 1, wherein the mold assembly is part of an injection molding system.

11. The mold assembly of claim 1, wherein at least a recess of the plurality of recesses comprises a half pyramidal geometry.

12. A method of forming a microneedle array, the method comprising:
heating a polymer to provide a molten polymer; and
causing the molten polymer to move into a mold assembly and through a plurality of perforated layers disposed therein and into a plurality of recesses, wherein the perforated layers restrict flow of the molten polymer into at least the plurality of recesses and facilitates expulsion of trapped gases,
wherein the plurality of perforated layers restrict flow of the molten polymer by filling a volume within the mold assembly except for space corresponding to the plurality of recesses in the mold assembly.

13. The method of claim 12, wherein the plurality of perforated layers forms a mold insert disposed within the mold assembly.

14. The method of claim 12, wherein the plurality of recesses correspond to a configuration of a microneedle array.

15. A microneedle array formed by a method comprising:
heating a polymer to provide a molten polymer; and
causing the molten polymer to move through a plurality of perforated layers into a plurality of recesses, wherein the perforated layers restrict flow of the molten polymer and facilitate release of trapped gases from the plurality of recesses thereby allowing the molten polymer to flow therein.

16. The microneedle array of claim 15, wherein the plurality of recesses correspond to a configuration of a microneedle array.

17. The microneedle array of claim 15, wherein the plurality of perforated layers forms a base for the microneedle array as the molten polymer is caused to move through the plurality of perforated layers.
